(19) United States
(12) Patent Application Publication
(10) Pub. No.: US 2022/0259636 A1
(43) Pub. Date: Aug. 18, 2022

(54) MICROBIAL COLONY DETECTION SYSTEM

(71) Applicants: THE WAVE TALK, INC., Yuseong-gu, Daejeon (KR); KOREA ADVANCED INSTITUTE OF SCIENCE AND TECHNOLOGY, Yuseong-gu, Daejeon (KR)

(72) Inventors: Young Dug KIM, Seongnam-si, Gyeonggi-do (KR); YongKeun PARK, Yuseong-gu, Daejeon (KR); Seung Yun HAN, Yuseong-gu, Daejeon (KR)

(21) Appl. No.: 17/624,817

(22) PCT Filed: Jul. 3, 2020
(86) PCT No.: PCT/KR2020/008756

§ 371 Date: Jan. 4, 2022

(30) Foreign Application Priority Data
Jul. 5, 2019 (KR) 10-2019-0081530
Jul. 3, 2020 (KR) 10-2020-0081893

(51) Int. Cl. C12Q 1/06; G01N 15/14; G01N 15/02; C12M 1/34

(52) U.S. Cl. CPC C12Q 1/06; G01N 15/1475; G01N 2015/03; C12M 41/36; G01N 15/0205

(57) ABSTRACT

Provided is a method of detecting microbial colonies, the method including: irradiating light having coherence to a sample unit in which a sample is accommodated; detecting, by an image sensor, transmitted light passing through the sample unit to obtain a sample image in a time-series manner; analyzing the sample image by a controller after a preset time to determine the concentration level of colonies in the sample; and obtaining a spatial correlation of the coherence pattern of the sample image when the concentration level determined by the controller is a high concentration that is equal to or greater than a preset reference value, and determining information about the concentration of colonies in the sample based on the change of the spatial correlation of the coherence pattern over time.

### TECHNICAL FIELD

Embodiments of the present disclosure relate to a microorganism colony detecting system.

### BACKGROUND ART

In the food or medical fields, unintentional microorganisms are frequently generated, which causes problems. In order to identify whether such microorganisms are proliferated, conventionally, a culture-type counting method using a medium has been used as a microorganism detecting system. For example, as a microorganism counting method, the method of counting the number of colonies of the microorganisms proliferated using an agar medium is used. On the other hand, instead of the method of visually counting the number of colonies generated on an agar medium, etc., a method of counting the number of colonies by data processing the image data of the medium to be counted of which an image is captured by using a CCD camera or the like has also been recently proposed.

However, in the case of these counting methods, the number of microorganisms cannot be directly counted, and instead, since microorganisms should be cultured until the colonies are identifiable with the naked eyes, at least one day is needed for the counting.

### DESCRIPTION OF EMBODIMENTS

### Technical Problem

The present disclosure provides a system for rapidly detecting microbial colonies from a sample image.

### Technical Solution to Problem

An embodiment of the present disclosure provides a method of detecting microbial colonies, the method including: irradiating light having coherence to a sample unit in which a sample is accommodated; detecting, by an image sensor, transmitted light passing through the sample unit to obtain a sample image in a time-series manner; analyzing the sample image by a controller after a preset time to determine the concentration level of colonies in the sample; and obtaining a spatial correlation of the coherence pattern of the sample image when the concentration level determined by the controller is a high concentration equal to or greater than a preset reference value, and determining information about the concentration of colonies in the sample based on the change of the spatial correlation of the coherence pattern over time.

### Advantageous Effects of Disclosure

The microbial colonies detecting system according to embodiments of the present disclosure can detect colonies in a sample within a short time. Since the degree of coherence pattern formation is affected only by colony formation, the microbial colonies detecting system may not be sensitive to vibration or external noise. In addition, the microbial colonies detecting system is not affected even when the culture substance of the sample unit is unstable. Accordingly, the microbial colonies detecting system enables fast and accurate measurements. In addition, since the microbial colonies detecting system performs the analysis by reflecting the area occupied by the colonies on the surface, even with a small number of colonies, the observation can be made after a certain period of time, and a single colony can be measured within a short time.

### BRIEF DESCRIPTION OF DRAWINGS

FIG. 1 shows a conceptual diagram schematically illustrating a microbial colonies detecting system according to an embodiment of the present disclosure.

FIG. 2 is a view showing another embodiment of the microbial colonies detecting system of FIG. 1.

FIG. 3 shows a flowchart sequentially illustrating a method of detecting microbial colonies according to an embodiment of the present disclosure.

FIG. 4 is a view for explaining a method of irradiating light to a sample unit.

FIG. 5 shows a conceptual diagram for explaining a first condition.

FIGS. 6 to 8 show diagrams for explaining the principle of the microbial colony detecting system according to an embodiment of the present disclosure to determine the concentration information of the high-concentration sample.

FIG. 9 shows an image of the colony growth degree in each concentration sample.

FIG. 10 shows a view for explaining the principle of performing an antimicrobial susceptibility test using a microbial colony detecting system according to embodiments of the present disclosure.

### MODE OF DISCLOSURE

An embodiment of the present disclosure provides a method of detecting microbial colonies, the method including: irradiating light having coherence to a sample unit in which a sample is accommodated; detecting, by an image sensor, transmitted light passing through the sample unit to obtain a sample image in a time-series manner; analyzing the sample image by a controller after a preset time to determine the concentration level of colonies in the sample; and obtaining a spatial correlation of the coherence pattern of the sample image when the concentration level determined by the controller is a high concentration equal to or greater than a preset reference value, and determining information about the concentration of colonies in the sample based on the change of the spatial correlation of the coherence pattern over time.

In an embodiment of the present disclosure, in the determining of the concentration level of colonies in the sample, when the number of colonies detected per unit area of the sample image is greater than or equal to the preset reference value, the concentration level of colonies is determined as high concentration, and when the number of colonies detected per unit area of the sample image is less than the preset reference value, the concentration level of colonies is determined as low concentration.

In an embodiment of the present disclosure, in the determining of the concentration level of colonies in the sample, when the degree of overlapping of single colonies detected in the sample image is greater than or equal to the preset reference value, the concentration level of colonies is determined as high concentration, and when the number of colonies detected per unit area of the sample image is less than the preset reference value, the concentration level of colonies is determined as low concentration.

In an embodiment of the present disclosure, in the determining of the concentration level of colonies in the sample, when the concentration level determined by the controller is less than a preset reference value, that is, a low concentration, the image of single colonies in the entire area of the sample image is obtained to count the number of colonies in the sample.

An embodiment of the present disclosure provides a microbial colonies detecting system including a sample unit for accommodating a sample, a light source that irradiates light having coherence toward the sample of the sample unit, an image sensor for obtaining a sample image in time series by detecting transmitted light passing through the sample unit, and a controller which analyzes the sample image after a preset time to determine the concentration level of colonies in the sample, obtains a spatial correlation of the coherence pattern of the sample image when the determined concentration level is equal to or greater than a preset reference value, that is, a high concentration, and determines the concentration information of colonies in the sample based on the time-dependent change of the spatial correlation of the coherence pattern.

In an embodiment of the present disclosure, the controller may determine colonies to have a high concentration when the number of colonies detected per unit area of the sample image is greater than or equal to a preset reference value, and may determine colonies to have a low concentration when the number of colonies detected per unit area of the sample image is less than the preset reference value.

In an embodiment of the present disclosure, the controller may determine colonies to have a high concentration when the overlapping degree of single colonies detected in the sample image is greater than or equal to a preset reference value, and may determine colonies to have a low concentration when the overlapping degree of single colonies detected in the sample image is less than the preset reference value.

In an embodiment of the present disclosure, when the determined concentration level is less than a preset reference value, that is, the low concentration, the controller may count the number of colonies in the sample by detecting images of single colonies in the entire area of the sample image.

Other aspects, features, and advantages other than the features will be apparent from the following drawings, claims, and detailed descriptions of the disclosure.

### Mode of Disclosure

Hereinafter, the following embodiments will be described in detail with reference to the accompanying drawings, and in the following description with reference to the drawings, like or corresponding components are denoted by like reference numerals, and redundant descriptions thereof will be omitted.

Since the present embodiments may apply various changes, specific embodiments are illustrated in the drawings and will be described in detail in the detailed description. The effects and features of the embodiments and methods of achieving the same will become apparent with reference to the following descriptions in detail with reference to the drawings. However, the embodiments are not limited to the embodiments described below, but may be implemented in various forms.

In the following embodiments, the terms “first” and “second” are not limited and are used to distinguish one component from other components.

In the following embodiments, a singular expression includes a plurality of expressions unless defined otherwise in context.

In the following embodiments, the terms such as “include” or “have” indicate that a feature or a component described in the specification is present, and do not preclude a possibility that one or more other features or components are added.

In the following embodiments, when a unit, an area, a component, etc. is located on or above another part, the present disclosure includes not only a case where the unit, the area, the component, etc. are located directly above the other part, but also a case where other units, other areas, other component, etc. may be located therebetween.

In the following embodiments, unless the terms “connecting” or “coupling” are clearly different in context, the terms “connecting” or “coupling” do not necessarily mean direct and/or fixed connection or coupling of two members, but do not exclude a member located between the two members.

It means that a feature or a component described in the specification is present, and does not preclude in advance a possibility that one or more other features or components are added.

In the drawings, for convenience of description, the size of the components may be exaggerated or reduced. For example, the size and thickness of each configuration shown in the drawings are arbitrarily shown for convenience of description, and thus the following embodiments are not necessarily limited to those shown in the drawings.

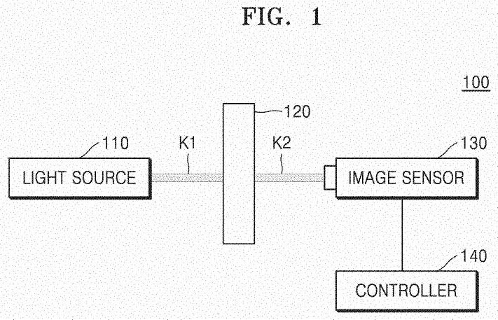

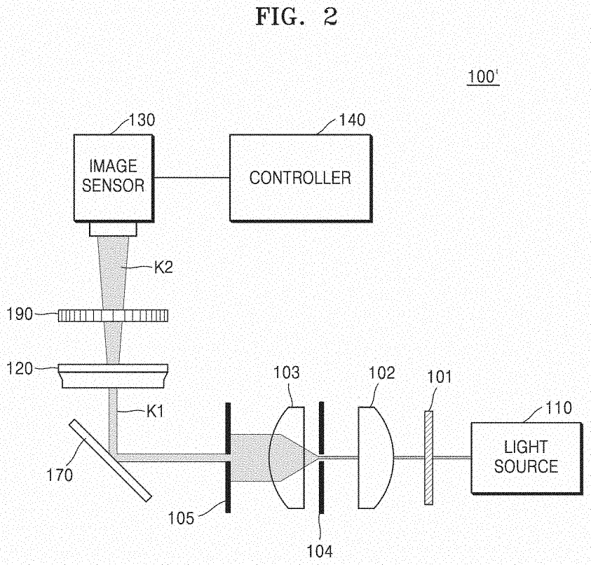

FIG. 1 shows a conceptual diagram schematically illustrating a microbial colonies detecting system according to an embodiment of the present disclosure, and FIG. 2 is a view showing another embodiment of the microbial colonies detecting system of FIG. 1.

Referring to FIGS. 1 and 2, microbial colony detecting systems 100 and 100′ according to embodiments of the present disclosure may include a light source 110, a sample unit 120, an image sensor 130, and a controller 140. The microbial colonies detecting system 100 of FIG. 1 includes the minimal configuration of the present disclosure, and the microbial colonies detecting system 100′ of FIG. 2 is a modified embodiment based on the microbial colony detecting system 100 of FIG. 1. Accordingly, redundant descriptions of the same configuration will be omitted.

The microbial colony detecting systems 100 and 100′ according to embodiments of the present disclosure is characterized in that a measurement object that changes in time series is detected using light. For example, the microbial colonies detecting systems 100 and 100′ may be a system for detecting a measurement object that grows over time, such as microorganisms. However, the present disclosure is not necessarily limited to detecting microorganisms, and any measurement object of which shape, concentration, or size changes in time series can be detected. Hereinafter, for convenience of description, the detection of microbial colonies will be mainly described.

The microbial colonies detecting systems 100 and 100′ have the technical concept of irradiating light to the sample unit 120 for culturing microorganisms and detecting the emitted light by using the image sensor 130 to detect or count colonies in the sample unit 120.

The light source 110 may irradiate light having coherence toward the sample. The light source 110 may be any type of source devices capable of generating light, and may be a laser capable of irradiating light having a specific wavelength band. For example, the light source 110 may be a laser that outputs light in a wavelength band of 532 nm.

For example, in order to form a speckle in a sample, a laser having a good coherence may be used as the light source 110. In this case, as a spectral bandwidth of a light source, determining the coherence of a light source, is reduced, the measurement accuracy may be increased. That is, as a coherence length increases, measurement accuracy may increase. Accordingly, a laser beam, of which the spectral bandwidth of the light source 110 is less than the predefined reference bandwidth, may be used as the light source 110, and the measurement accuracy may be increased as the spectral bandwidth of the light source is shorter than the reference bandwidth. For example, the spectral bandwidth of the light source 110 may be set to maintain Equation 1.

Spectral bandwidth<5 nm [Equation 1]

According to Equation 1, in order to measure the pattern change of the laser speckle, the spectral bandwidth of the light source 110 may be maintained to be less than 5 nm when light is irradiated into the sample at every reference time.

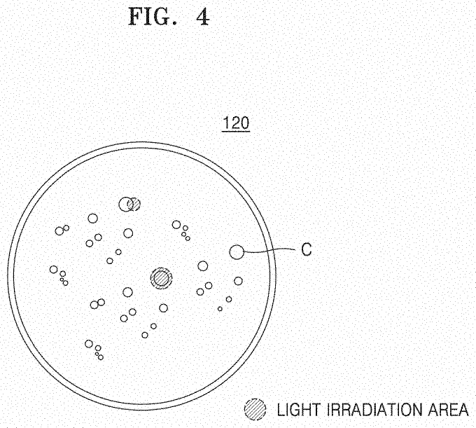

As shown in FIG. 2, when the microbial colonies detecting system 100′ according to another embodiment irradiates the light generated from the light source 110 to the sample unit 120, there is a need to increase the optical properties to increase analysis accuracy. To this end, the microbial colonies detecting system 100′ may include one or more optical elements between the light source 110 and the sample unit 120. For example, the optical element may include a filter 101, one or more optical lens including a first optical lens 102 and a second optical lens 103, a pinhole member 104, a mirror 107, an aperture 105, and the like. The microbial colonies detecting system 100′ may control the irradiation area of the light irradiated to the sample unit 120 by using the optical elements. For example, the microbial colonies detecting system 100′ may control the irradiation area of light to irradiate the light in an area larger than the size of a single colony by using the optical elements. A detailed description thereof will be described with reference to FIG. 4.

The filter 101 may perform a function to control the intensity of light irradiation or to remove noise, and may be, for example, an neutral density (ND) filter. One or more optical lenses including the first optical lens 102 and the second optical lens 103 may perform a function to guide the path of light. As shown in the figure, the first optical lens 102 may be disposed between the pinhole member 104 and the filter 101 to guide the path of light to the opening of the pinhole member 104, and the second optical, and the second optical lens 103 may be disposed between the pinhole member 104 and the sample unit 120 to control the size of light to collimate the light into parallel rays. The pinhole member 104 functions as a spatial frequency filter and may uniformly adjust a wavefront of light. In addition, the aperture 105 may perform a function of guiding only a part of the collimated light extended from the second optical lens 103, which is a collimator, into the sample unit 120.

The sample unit 120 may accommodate a sample to be measured. The sample unit 120 may be any type of container capable of accommodating a sample, for example, an agar plate containing a culture medium for culturing microorganisms. The sample may be accommodated in the sample unit 120 by various methods, and In an embodiment, may be accommodated in the sample unit 120 by a pouring method. The microbial colonies detecting systems 100 and 100′ may measure a sample in a transparent state, but may also measure a sample having turbidity or a sample having an opaque state such as meat or ham. The sample unit 120 may include a culture substance for culturing microorganisms. The culture substance corresponds to the type of microorganism to be counted and may include a substance capable of effectively culturing the microorganism.

The medium containing the culture substance used for culture should properly satisfy the requirements of specific microorganisms. Various microbial culture media are disclosed in, for example, the document (“Manual of Methods for General Bacteriology” by the American Society for Bacteriology, Washington D.C., USA, 1981.). These media contain various carbon sources, nitrogen sources and trace element components. Carbon sources include: fats, such as glucose, lactose, sucrose, fructose, maltose, carbohydrates, such as starch and fiber; oils, such as soybean oil, sunflower oil, castor oil, and coconut oil; fatty acids such as palmitic acid, stearic acid and linoleic acid; alcohols such as glycerol and ethanol; and organic acids such as acetic acid, and these carbon sources may be used alone or in combination, but are not limited thereto. Nitrogen sources include organic nitrogen sources such as peptone, yeast extract, gravy, malt extract, corn steep liquor (CSL), and bean flour and inorganic nitrogen sources such as urea, ammonium sulfate, ammonium chloride, ammonium phosphate, ammonium carbonate, and ammonium nitrate, and these nitrogen sources may be used alone or in combination, but are not limited thereto. The medium may additionally contain, as a phosphoric acid source, potassium dihydrogen phosphate, dipotassium hydrogen phosphate, an corresponding sodium-containing salts, but is not limited thereto. In an embodiment, the medium may contain a metal such as magnesium sulfate or iron sulfate, and amino acids, vitamins and suitable precursors may be added thereto.

In an embodiment, in order to maintain the aerobic condition of the culture medium, oxygen or a gas containing oxygen (for example, air) may be loaded into the culture medium. The temperature of the culture may be generally 20° C. to 45° C., for example, 25° C. to 40° C. In order to control the temperature of the culture, the sample unit 120 may be connected to a heating element.

In an embodiment, the sample unit 120 may include a lid member (not shown). At this time, a heating element may be provided to the lid member (not shown) to perform a function of evaporating moisture in the sample unit 120.

The image sensor 130 is disposed on the path of light emitted from the sample unit 120 and measures an optical image which is emitted, that is, a sample image. The image sensor 130 senses transmitted light K2 passing through the sample unit 120 to obtain a sample image in the time-series manner. For example, the image sensor 130 may be a charge-coupled device (CCD) camera. The microbial colonies detecting systems 100 and 100′ according to embodiments of the present disclosure may detect, with the same configuration, of only a low concentration microorganism but also a high concentration microorganism. In other words, the image sensor 130 may measure the optical image emitted from the sample unit 120 and transmit the same to the controller 140. In this regard, in order to obtain both the sample image for the low-concentration sample and the high-concentration sample, the image sensor 130 may be spaced apart from the sample unit 120 by a preset distance. This will be described later.

Meanwhile, the microbial colonies detecting system 100′ may include a polarizer 190 on the path of transmitted light K2 emitted from the sample unit 120 and incident to the image sensor 130, to remove unnecessary external reflected light.

The controller 140 may analyze the sample image obtained from the image sensor 130 to count the number of colonies in the sample or to determine concentration information. Hereinafter, a method of counting the number of colonies in a sample or determining concentration information, performed by the controller 140, will be described in detail with reference to the drawings.

The microbial colonies detecting system 100 having the configuration described above may irradiate light K1 to the sample unit 120 for culturing microorganisms and may detect the transmitted light K2 with the image sensor 130 to detect or count colonies in the sample unit 120. At this time, the colonies act as a diffuser with respect to the incident light K1. That is, before colonies grow, the light K1 incident to the sample unit 120 does not have a diffusion medium and is emitted as it is, but when the colonies grow, the light incident to the sample unit 120 is scattered due to the colonies and thus a coherence pattern or a diffraction pattern may be formed.

The microbial colonies detecting system 100 captures the image of the coherence pattern or diffraction pattern formed by colonies by using the image sensor 130, and analyzes the pattern displayed in the captured optical image to detect colonies or to count the number of colonies. The microbial colonies detecting system 100 may detect colonies or count the number of colonies regardless of the concentration of microorganisms in the initial sample.

Conventionally, it is said that counting about 30 to 300 colonies is the most reliable when bacteria is measured in a medium test. Thus, it is common to dilute according to the initial concentration and then the medium test is performed therewith. In this case, however, when the initial concentration of microorganisms is not known, the experiment has to be performed with various diluted samples, resulting in time and cost loss. The present disclosure is intended to solve this problem, and has the advantage of being able to measure using a single device regardless of the concentration of the initial sample through the method described below.

The term “microorganism” used herein refers to a prokaryotic or eukaryotic microorganism having the ability to produce useful target substances such as L-amino acids. For example, microorganisms with increased intracellular ATP concentrations may be Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp. or fungi or yeast. In an embodiment, the microorganism may be a microorganism of the Escherichia species.

In an embodiment, the microorganism may be selected from bacteria selected from Staphylococcus sp., staph Coagulase negative sp., Staph. aureus, Streptococcus spp., Streptococcus viridans group, Enterococcus spp., Corynebacterium spp., Aerococcus spp., Micrococcus spp., Peptostreptococcus spp., Lactococcus spp., Leuconostoc spp., Tothia spp., Gemella spp., Alcaligenes spp., Alternaria spp. Flavobacterium spp., Bacillus spp., Achromobacter spp., Acinetobacter spp., Actinobacillus spp., Alcaligenes spp., Campylobacter spp., Edwardsiella spp., Ehrlicia spp., Enterobacter spp., Ewingella spp., Flavobacteria sp., Hafnia spp., Klebsiella spp. Kluyvera spp., Legionella spp., Morxella spp., Morganella spp., Neisseria spp., Pasteurella spp., Prevotella spp., Proteus spp., Providencia spp., Pseudomonas spp. Rahnella spp., Salmonella spp., Serratia spp., Shigella spp., Sphingobacterium spp. Vibrio spp., Yersinia spp., Neisseria spp., Kingella spp. Cardiobacterium sp., non-Tuberculosis mycobacteria (NTB), Mycobacterium tuberculosis, and Mycobacterium avium. In an embodiment, the microorganism may be E. coli. However, the technical concept of the present disclosure is not limited thereto, and other microorganisms may be further included.

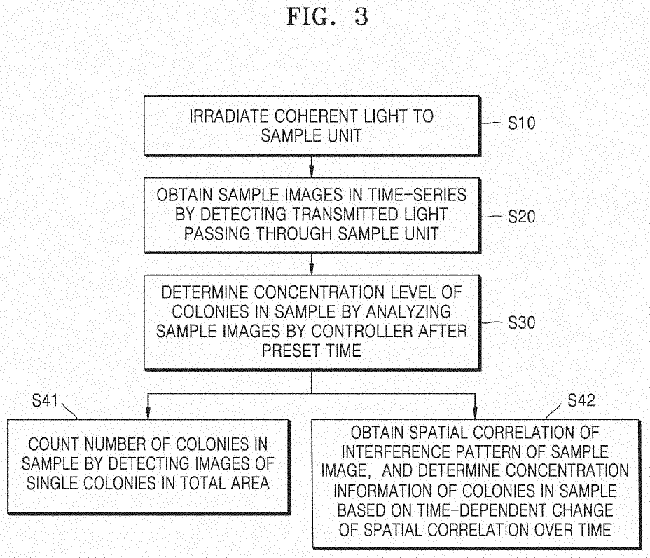

FIG. 3 shows a flowchart sequentially illustrating a method of detecting microbial colonies according to an embodiment of the present disclosure, and FIG. 4 is a view for explaining a method of irradiating light to the sample unit 120.

Referring to FIGS. 1, 3, and 4, the method of detecting microbial colonies according to an embodiment of the present disclosure includes irradiating coherent light to the sample unit 120 by using the light source 110 (S10). In this case, the microbial colonies detecting system 100 may control the irradiation area of the incident light K1 irradiated from the light source 110 to provide the same to the sample unit 120.

The microbial colonies detecting system 100 may control the irradiation area of light by using optical elements to be larger than the size of at least one single colony C or to include at least a portion of one single colony C and a portion of the surrounding area. Here, defined with respect to the single colony C, the surrounding area refers to an area in which another single colony is formed or an area in which a single colony is not formed. In addition, the microbial colonies detecting system 100 may provide light generated from the light source 110 to the sample unit 120 by using optical elements in such a manner that the irradiation area of the light is large enough to cover the entire area of the sample unit 120.

The microbial colonies detecting system 100 may irradiate light to the sample unit 120 while the irradiation area of light varies. For example, the microbial colonies detecting system 100 may control: the irradiation area of light to cover the entire area of the sample unit 120 when the concentration level of colonies in the sample is schematically determined, and thereafter; when the concentration information of colonies is determined, the irradiation area of light to a size large enough to cover one single colony C and the surrounding area.

Next, according to the method of detecting microbial colonies, the image sensor 130 may obtain the sample image in the time-series manner by sensing the transmitted light K2 passing through the sample unit 120 (S20). The image sensor 130 may measure the optical image emitted from the sample unit 120 and transmit the same to the controller 140. In this case, to obtain both an optical image for a low-concentration sample and an optical image for a high-concentration sample, the image sensor 130 may be disposed apart from the sample unit 120 by a preset distance.

Specifically, the preset distance may have such a range that satisfies a first condition that the image sensor 130 can sufficiently distinguish a single colony from a low-concentration sample, and a second condition that the speckle size is larger than the pixel size in a high-concentration sample.

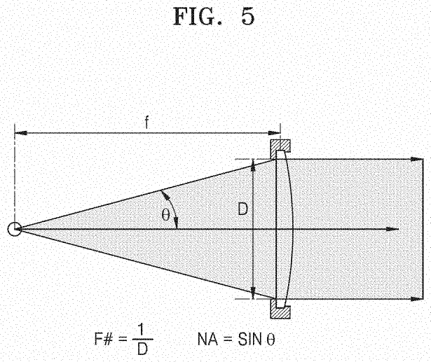

FIG. 5 shows a conceptual diagram for explaining a first condition.

Referring to FIGS. 1, 3 and 5, the first condition may be derived from the following Equation 2 by applying the Rayleigh criterion for lateral resolution.

$$r = \frac{1.22 λ}{2 n sin θ} = \frac{0.61 λ}{NA} \qquad [ Equation 2 ]$$

Here, r is the distinguishable minimum distance, n is the refractive index (assuming as being air, n=1), λ is the laser wavelength (for example, 532 nm), and θ is the maximum angle at which light arriving at the image sensor is tilted with respect to the vertical axis.

When two colonies are spaced apart from each other by a distance in the sample, it is possible to distinguish the two colonies only when the following condition in Equation 3 is satisfied: Rayleigh criterion<a.

$$\frac{0.61 λ}{sin θ} < a \qquad [ Equation 3 ]$$

At this time, assuming that about 100 cfu is seeded in an area of 1 cm² (when 10⁵ cfu/ml is seeded by 5 μl), the average distance between colonies is as follows.

$${a}_{avg} = \sqrt{\frac{1 {cm}^{2}}{100}} ≅ 1 mm$$

In addition, in the case where the distance between the image sensor 130 and the sample is L, and the area on which microorganisms are sprayed is 1 cm² (D=1 cm in width and length), NA is as follows.

$$sin θ = \frac{D}{2 L} = \frac{1 cm}{2 L} \qquad [ Equation 4 ]$$

Here, when a_{avg} and sine are substituted in Equation 3, the distance range between the image sensor 130 and the sample unit 120, specifically, the image sensor 130 and the sample, in the first condition, may be set as follows:

$$L < \frac{{a}_{avg} D}{1.22 λ} = \frac{( 1 mm ) ( 1 cm )}{1.22 ( 532 nm )} = 15.4 m .$$

Meanwhile, the distance range satisfying the second condition is as follows.

Specklesize>Pixelsize [Equation 5]

The pixel size of the image sensor 130 may be a value determined by the image sensor 130. For example, the image sensor 130 may be a commercially available lumenera camera, and the lumenera camera may have a pixel size of 4.54 μm (based on the specification sheet).

The speckle size (d) is determined by NA and the wavelength, and may be expressed as in Equation 6 below.

$$d = \frac{λ}{4 NA} \qquad [ Equation 6 ]$$

Similarly, by substituting Equation 6 into Equation 5, the following result may be obtained.

$$pixel size < \frac{L λ}{2 D}$$

$$4.54 μm < \frac{L ( 532 nm )}{2 ( 1 cm )}$$

$$L > 4.3 cm$$

The image sensor 130 may be disposed apart from the sample unit 120 by a preset distance range derived as described above. The preset distance range is as follows.

4.3 cm<L<15.4 m

However, the values derived by the above process correspond to a single embodiment, and the present disclosure is not limited by the above result values.

Next, according to the microbial colonies detection method, after the preset time, the concentration level of colonies in the sample may be measured by analyzing the sample image by the controller 140 (S30).

As described above, since microorganisms may perform the function of a diffuser only when they grow into a single colony, a preset time for the growth into a single colony is required. The preset time may vary depending on the type of microorganism to be measured.

The controller 140 may determine, after a preset time, the concentration level of colonies in the sample by using the sample image captured by the image sensor 130. In an embodiment of the present disclosure, the controller 140 may determine colonies to have a high concentration when the number of colonies detected per unit area of the optically shown sample image is greater than or equal to a preset reference value, and may determine colonies to have a low concentration when the number of colonies detected per unit area of the optically shown sample image is less than the preset reference value. Here, the low concentration may refer to a concentration at which single colonies do not overlap each other and may be distinguishable, and the high concentration may refer to a concentration at which single colonies overlap without being differentiated due to a large number of microorganisms. In this process, the controller 140 does not accurately count the number of colonies or analyze the concentration, but only performs a schematic analysis to determine whether the concentration level has a low concentration or a high concentration.

The controller 140 analyzes how many microbial colonies are optically visible in the sample image per unit area, and determines the concentration level of the microbial colonies. In this case, the controller 140 may perform analysis on the entire area of the sample image, but, in an embodiment, the concentration level may be determined using the unit-area image sampled from the sample image.

The controller 140 may use an image analysis method well known in the art to detect microbial colonies in a sample image. The image analysis method may be a method of identifying microbial colonies within a sample image based on one or more criteria including the target size, the visibility, the color, the surface quality, and the shape. For example, the controller 140 may detect microbial colonies by using the shading degree of each pixel of the sample image.

In an embodiment, the controller 140 may determine colonies to have a high concentration when the overlapping degree of single colonies detected in the sample image is greater than or equal to a preset reference value, and may determine colonies to have a low concentration when the overlapping degree of single colonies detected in the sample image is less than the preset reference value. The controller 140 may detect the edge of a single colony by using the image analysis method. Here, when the edges of single colonies do not overlap each other, the controller 140 may determine that the sample has the low concentration. In the case where the edges of single colonies overlap, when the overlapping degree is equal to or greater than a certain level, the controller 140 may determine that the sample has the high concentration. In this case, the controller 140 may determine the concentration level by averaging the overlapping degrees of single colonies included in a unit area instead of one single colony.

Next, the controller 140 according to the present disclosure may vary a method of analyzing the sample image according to whether the concentration level is a low concentration or a high concentration.

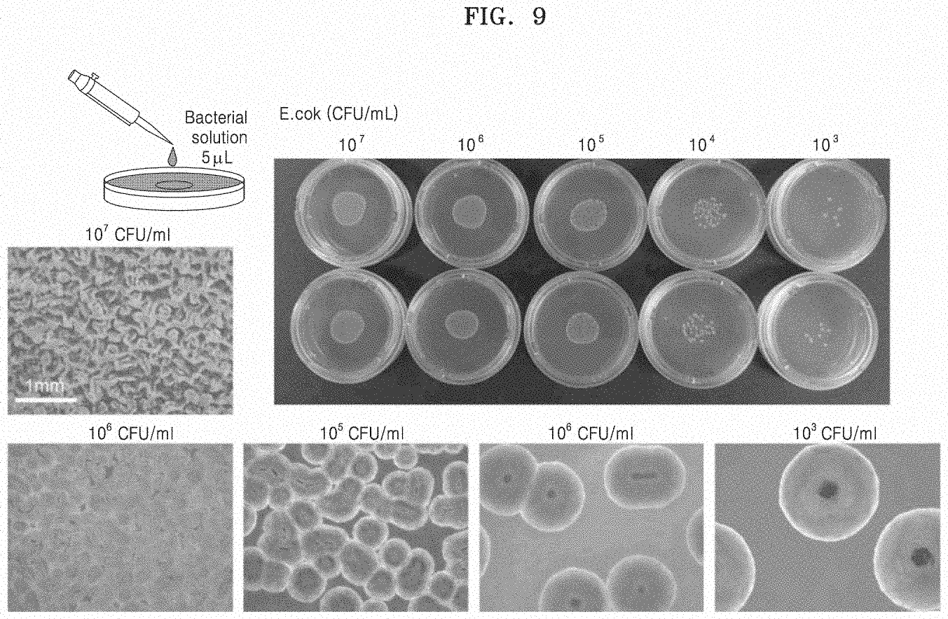

When the concentration is low (S41), the controller 140 may obtain images of single colonies in the entire area of the sample image and count the number of colonies in the sample. In the case of low-concentration samples such as 10³ cfu/ml, 10⁴ cfu/ml, and 10⁵ cfu/ml shown in FIG. 9, colonies grow non-overlapping to the extent that they are distinguishable from each other, so that the controller 140 may, as described above, count the number of colonies directly from the visual image. In other words, in the process of determining the concentration level, the controller 140 uses the sample image that is a visual image to roughly determine whether the concentration is low or high, and when it is determined that the concentration is low, the sample image, which is a visual image, is used again to count the total number of colonies. Similarly, the controller 140 may use an image analysis method, and unlike the previous process, the controller 140 may count the number of microbial colonies in the entire area.

When the concentration is high (S42), the controller 140 obtains the spatial correlation of the coherence pattern of the sample image, and based on the time-dependent change of the spatial correlation of the coherence pattern, collects information on the concentration of colonies in the sample. In the case of a high concentration, since it is impossible to visually count the number of microbial colonies, the microbial colonies detection method according to the present disclosure may obtain concentration information on the high concentration colonies through the following method.

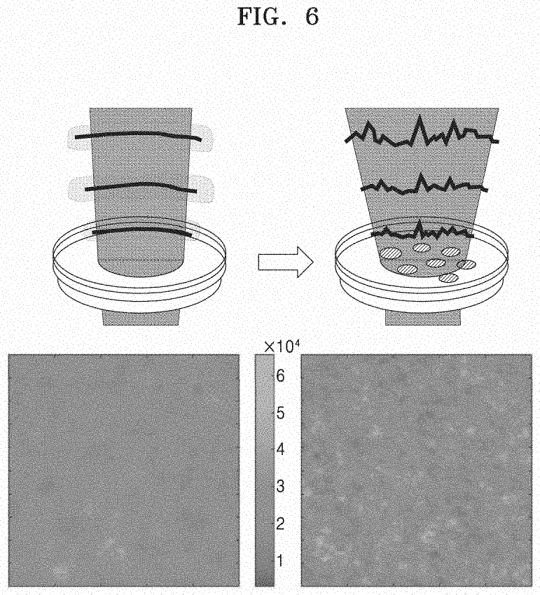

FIGS. 6 to 8 show diagrams for explaining the principle of the microbial colony detecting system 100 according to an embodiment of the present disclosure to determine the concentration information of the high-concentration sample, and FIG. 9 shows an image of the colony growth degree in each concentration sample.

Referring to FIGS. 6 to 9, the controller 140 may receive, from the image sensor 130, an optical image measured in the time-series manner, and may determine the density information of colonies in the sample from the optical image. At this time, the colonies act as a diffuser with respect to the incident light. That is, as illustrated in the left structure of FIG. 6, before colonies grow, the light incident to the sample unit 120 does not have a diffusion medium and is emitted as it is, but when the colonies grow, the light incident to the sample unit 120 is scattered due to the colonies and thus a coherence pattern may be formed.

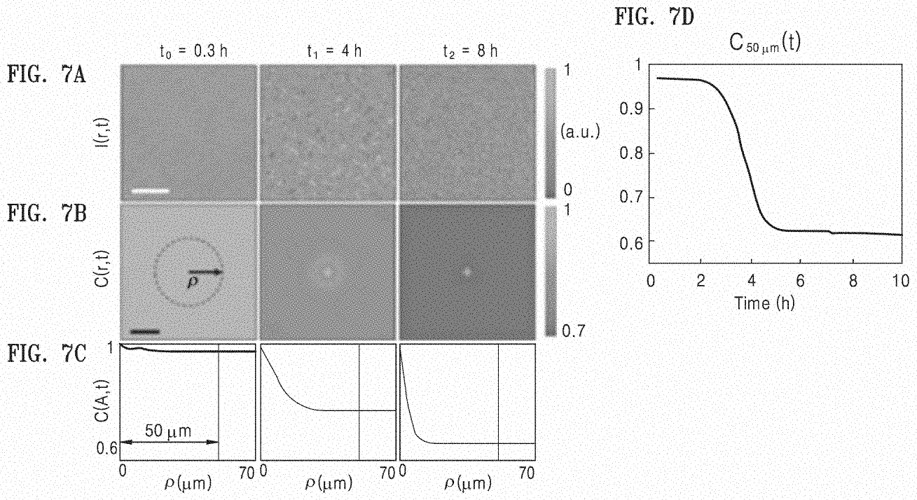

The controller 140 may obtain a spatial correlation of the coherence pattern. Here, the spatial correlation given by the following equation may be expressed as a certain range of number to evaluate how similar the brightness of a random pixel and the brightness of pixel separated by a distance r from the pixel are on the image obtained at time t ((b) of FIG. 7). The certain range may be in the range of −1 to 1. That is, the spatial correlation indicates the degree of correlation between an arbitrary pixel and other pixels. When the spatial correlation is 1, the correlation is positive; when the spatial correlation is −1, the correlation is negative; and when the spatial correlation is 0, there is no relationship. Specifically, before the coherence pattern is formed, the spatial correlation of the sample image shows a positive correlation close to 1 because the brightness is evenly emitted. However, once the coherence pattern is formed, the correlation value may be decreased close to 0.

Regarding the image sensor 130, the brightness measured at time tin the pixel located at r′=(x,y) is defined as l(r′,t), and the brightness of the pixel separated by r is defined as l(r′+r, t). When the spatial correlation is defined using these, it can be expressed by Equation 7.

$$C ( r , t ) = \frac{1}{{C}_{0} ( t )} \int \int I ( {r}^{′} + r , t ) I ( {r}^{′} , t ) {dr}^{′} \qquad [ Equation 7 ]$$

C₀(t) was used to adjust the range of Equation 7 to be −1 to 1. When the brightness l(r′,t) measured at time tin any pixel is equal to the brightness l(r′+r,t) of a pixel separated therefrom by a distance r, the spatial correlation is 1, and otherwise, the spatial correlation may be smaller than 1.

In an embodiment of the present disclosure, the spatial correlation may be expressed only as a function of time. To this end, the controller 140 may obtain the average of the spatial correlations for pixels separated by the same distance of r from any pixel as in Equation 8 below (refer to (b) of FIG. 7).

$$C ( ρ , t ) = \frac{1}{2 π} {\int }_{0}^{2 π} C ( r , t ) d θ \qquad [ Equation 8 ]$$

In an embodiment, the controller 140 may substitute the preset distance into Equation 8 to express the spatial correlation as a function of time. By using this function, the degree of formation of the coherence pattern is set to be within a range of 0 to 1 (refer to (d) of FIG. 7).

The controller 140 may also distinguish a foreign substance and microbial colonies in the sample through a change in the pattern of the sample image over time. In the case of foreign substances, there is no change in the image over time, but in the case of microbial colonies, since the image thereof has changes in, for example, the shape and the size, over time, the microbial colonies detecting system 100 can distinguish foreign substances from microbial colonies.

Meanwhile, the controller 140 may determine the concentration information of microbial colonies by using spatial correlation as described below. The spatial correlation may be obtained in such a manner that two identical images overlapping are generated by using a single image, and one of the two images is shifted in one direction by a preset distance, and then how similar two adjacent pixels of each of the shifted image and the unshifted image are, is analyzed. Here, the spatial correlation is a measure of how uniform the image is. In the case where a coherence pattern is formed due to colonies, the similarity of two adjacent pixels is decreased due to small coherence patterns, so that the value of spatial correlation also is decreased.

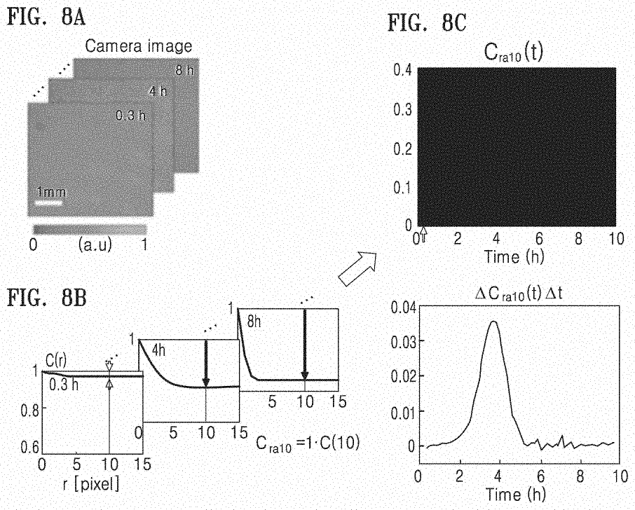

The spatial correlation coefficient is changed depending on the shifting distance r (see (b) of FIG. 8), and within a certain distance range, the spatial correlation coefficient is decreased as the shifting distance r is increased, and when exceeding the certain distance range, the spatial correlation coefficient may have an almost constant value. Accordingly, in order to obtain a more meaningful spatial correlation, the controller 140 may acquire the spatial correlation by shifting the image by a preset distance or more. At this time, the preset distance, r, depends on the speckle size, and the controller 140 may obtain spatial correlation by shifting the image by a pixel larger than the speckle size when expressed in units of pixels. For example, the preset distance may be the distance corresponding to at least 3 pixels or more.

Meanwhile, the controller 140 may obtain not only the spatial correlation as described above but also a temporal correlation of a coherence pattern of a measured sample image, and detect microorganisms based on the obtained temporal correlation. The controller 140 may calculate a temporal correlation coefficient of images by using the image information of the coherence pattern measured in time series, and, based on the temporal correlation coefficient, microbial colonies in the sample may be detected.

The controller 140 may detect microbial colonies through analysis in which the calculated temporal correlation coefficient falls below the preset reference value. Through the temporal correlation analysis of the coherence pattern formed by the minute movement of the microorganism, the microbial colonies detecting system 100 may estimate the presence or concentration of the microorganism even in a state in which colonies are not formed in the sample.

For the analysis, the microbial colonies detecting system 100 according to an embodiment of the present disclosure may further include a multiple-scattering amplification element for amplifying the number of times that light incident on the sample unit 120 is multiple scattered within the sample. For example, a multiple-scattering amplification element (not shown) may be provided on the path of light between the light source 110 and the sample unit 120 or between the sample unit 120 and the image sensor 130 to amplify the number of multiple scatterings of light. The multiple-scattering amplification element (not shown) may be configured to be mountable on or detachable from a system, and may be used as needed.

That is, the controller 140 may analyze the microorganism in the sample by using together the temporal correlation of the coherence pattern measured in the time-series manner as well as the spatial correlation of images of the coherence pattern formed by colonies.

Through the above configuration, the microbial colonies detecting system according to embodiment of the present disclosure may detect colonies in a sample for a short time. Since the degree of coherence pattern formation is affected only by colony formation, the microbial colonies detecting system may not be sensitive to vibration or external noise. In addition, the microbial colonies detecting system is not affected even when the culture substance of the sample unit is unstable. Accordingly, the microbial colonies detecting system enables fast and accurate measurements. In addition, since the microbial colonies detecting system performs the analysis by reflecting the area occupied by the colonies on the surface, even with a small number of colonies, the observation can be made after a certain period of time, and a single colony can be measured within a short time.

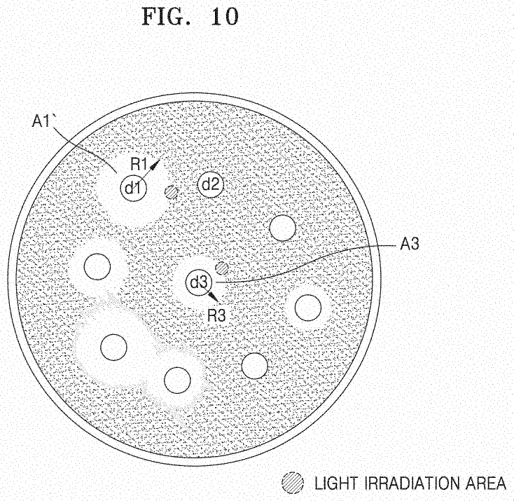

FIG. 10 shows a view for explaining the principle of performing an antimicrobial susceptibility test using a microbial colony detecting system according to embodiments of the present disclosure.

Referring to FIG. 10, the microbial colonies detecting system 100 may be utilized for an antimicrobial susceptibility test. An antimicrobial susceptibility test may be performed by a colony counting method and a disk diffusion method. According to the colony counting method, the solution of microorganism, which has reacted with the specimen for a certain period of time, is smeared on a nutrient agar, and then, the microorganism is allowed to grow for a certain period of time, and by counting the microbial colonies, the antimicrobial activity of a specimen is measured. This method may use the counting method for microbial colonies or the method of determining concentration information, as described above.

The antimicrobial susceptibility test method shown in FIG. 10 is performed by the disk diffusion method. According to the disk diffusion method of the related art, a specimen is placed on a nutrient agar medium on which a certain amount of microorganism is smeared, and then, the microorganism is allowed to grow for a certain period of time to measure the size of the microbial growth inhibition zone around the specimen due to the influence of the specimen. By using the disk diffusion method, the qualitative antibacterial activity of the specimen may be obtained. In conventional cases, the size of the microbial growth inhibition zone is directly measured by a human hand. Accordingly, the accuracy is low.

Similar to the method described above, according to the microbial colonies detecting system according to embodiments of the present disclosure, light is irradiated to a culture plate on which an antibiotic disk is disposed, and transmitted light is detected to evaluate the antibiotic sensitivity from a sample image obtained. Specifically, the first antibiotic disk d1 to the third antibiotic disk d3 containing different antibiotics may be disposed on a culture plate.

As illustrated in the drawing, the size of the microbial growth inhibition zone of the second antibiotic disk d2 is hardly increased, while a microbial growth inhibition zone A1 of the first antibiotic disk d1 and a microbial growth inhibition zone A3 of the third antibiotic disk d3 are increased in size. At this time, the size of the first microbial growth inhibition zone A1 may be different from the size of the third microbial growth inhibition zone A3.

The microbial colonies detecting system may scan light from the center of each microbial growth inhibition zone to the edge thereof, based on each antibiotic disk. In the case of the light incident on a microbial growth inhibition zone, the emitted light may have uniform brightness due to the absence of the microbial growth inhibition zone. Accordingly, during the scanning on the microbial growth inhibition zone, the change in emitted light may not occur. In contrast, when light is incident to the edge of the microbial growth inhibition zone, light may be irradiated to both an area in which microorganisms exist and an area in which microorganisms do not exist. In this case, an coherence pattern may be formed due to different optical properties of the region in which the microorganisms are present and the region in which the microorganisms do not exist.

The microbial colonies detecting system can quickly and accurately calculate the size of the microbial growth inhibition zone by sensing the change in the coherence pattern during scanning around the antibiotic disk and the surrounding area thereof.

Hereinbefore, the present disclosure has been described using embodiments. It will be appreciated by those skilled in the art that the present disclosure can be implemented in a modified form in a range that does not deviate from the essential characteristics of the present disclosure. Therefore, the disclosed embodiments should be considered in terms of explanation, not in a limited aspect. The scope of the present disclosure is not described above, but is shown in the claims, and all differences within the scope of the present disclosure should be construed as being included in the present disclosure.

### INDUSTRIAL APPLICABILITY

According to an embodiment of the present disclosure, a microbial colonies detecting system is provided. Also, embodiments of the present disclosure may be applied to an apparatus for detecting impurities or microorganisms, which are used for industrial purposes.

1. A method of detecting a microbial colony, the method comprising:
irradiating light having coherence to a sample unit in which a sample is accommodated;
obtaining, by an image sensor, a sample image in a time-series manner by sensing transmitted light passing through the sample unit;
determining a concentration level of colonies in the sample by analyzing the sample image by the controller, after a preset time; and
obtaining a spatial correlation of the coherence pattern of the sample image, and based on a time-dependent change of a spatial correlation of the coherence pattern, determining the concentration information of colonies in the sample, when the concentration level determined by the controller is a high concentration equal to or greater than a preset reference value.

2. The method of claim 1, wherein,
in the determining of the concentration level of colony in the sample,
when the number of colony to be detected per unit area of the sample image is greater than or equal to the preset reference value, it is determined the concentration level is a high concentration, and when the number of colony to be detected per unit area of the sample image is less than the reference value, it is determined the concentration level is a low concentration.

3. The method of claim 1, wherein
in the determining of the concentration level of colonies in the sample,
when a degree of overlapping of single colonies detected in the sample image is greater than or equal to the preset reference value, it is determined the concentration level is a high concentration, and when a degree of overlapping of single colonies detected in the sample image is less than the reference value, it is determined the concentration level is a low concentration.

4. The method of claim 1, wherein
in the determining of the concentration level of colonies in the sample,
when the determined concentration level is a low concentration that is less than a preset reference value, the controller counts the number of colonies in the sample by detecting images of single colonies in the entire area of the sample image.

5. A system for detecting microbial colony, the system comprising:
a sample unit to accommodate a sample;
a light source to irradiate light having coherence toward the sample of the sample unit;
an image sensor to detect transmitted light passing through the sample unit to obtain sample images in a time-series manner; and
a controller which determines the concentration level of colony in the sample by analyzing the sample image after a preset time, obtains a spatial correlation of the coherence pattern of the sample image when the determined concentration level is a high concentration that is equal to or greater than a preset reference value, and determines the concentration information of colonies in the sample based on the time-dependent change of the spatial correlation of the coherence pattern.

6. The system of claim 5, wherein
when the number of colony to be detected per unit area of the sample image is greater than or equal to the preset reference value, the controller determines the concentration level as a high concentration, and when the number of colony to be detected per unit area of the sample image is less than the reference value, the controller determines the concentration level as a low concentration.

7. The system of claim 5, wherein
when a degree of overlapping of single colonies detected in the sample image is greater than or equal to a preset reference value, the controller determines the concentration level as a high concentration, and when a degree of overlapping of single colonies detected in the sample image is less than the preset reference value, the controller determines the concentration level as a low concentration.

8. The system of claim 5, wherein
the controller counts the number of colonies in the sample by detecting images of single colonies in the entire area of the sample image, when the determined concentration level is a low concentration that is less than a preset reference value.
